(19) United States
(12) Patent Application Publication
(10) Pub. No.: US 2026/0207439 A1
(43) Pub. Date: Jul. 23, 2026

(54) CARIOSTATIC RESTORATION COMPOSITION

(71) Applicant: IMAM ABDULRAHMAN BIN FAISAL UNIVERSITY, Dammam (SA)

(72) Inventors: B. Rabindran JERMY, Dammam (SA); Abdullah ALJAMI, Dammam (SA); Rasha ALSHEIKH, Dammam (SA)

(73) Assignee: IMAM ABDULRAHMAN BIN FAISAL UNIVERSITY, Dammam (SA)

(21) Appl. No.: 19/033,371

(22) Filed: Jan. 21, 2025

(51) Int. Cl. A61K 6/869; A61K 6/15; A61K 6/838; A61K 6/844

(52) U.S. Cl. CPC A61K 6/869; A61K 6/15; A61K 6/838; A61K 6/844

(57) ABSTRACT

A cariostatic restoration composition includes TiZSM-5 nanoparticles, silver, sodium hexametaphosphate, and a glass ionomer cement. The TiZSM-5 nanoparticles are mesoporous and include surfaces and mesopores. The silver is disposed on the surfaces and impregnated into the mesopores, and the sodium hexametaphosphate is disposed on the surfaces and impregnated into the mesopores. The amount of sodium hexametaphosphate is in a range from 40 to 160 wt. % of the weight of the TiZSM-5 nanoparticles and the amount of silver is in a range from 2 to 6 wt. % of the total weight of TiZSM-5 nanoparticles. The combined amount of sodium hexametaphosphate, silver, and TiZSM-5 nanoparticles is in a range from 0.1 to 10 wt. % of the weight of the glass ionomer cement.

### BACKGROUND

### Technical Field

The present disclosure is directed to a cariostatic restoration, and particularly directed to a cariostatic restoration composition, including Ag-impregnated TiZSM-5 zeolite glass ionomer restoration functionalized with sodium hexametaphosphate (SHMP).

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present disclosure.

Generally, cariostatic restoration, such as glass ionomer cements (GIC) is implemented for the management of coronal caries and root caries [See: Tonprasong W., et al., Impact of direct restorative dental materials on surface root caries treatment. Evidence based and current materials development: A systematic review, Jpn Dent Sci Rev, 2022 Nov., 58, 13-30]. The antibacterial effect has been reported to extend up to 20 micrometers to neighboring tissue. The GIC also has the property to chemically bond with tooth structure without a bonding agent [See: Lohbauer U., Dental Glass Ionomer Cements as Permanent Filling Materials?-Properties, Limitations Future Trends, Materials (Basel), 2009, 3, 1, 76-96]. Nonetheless, GIC is capable of producing fluoride, and it goes through an initial burst of fluoride, including during setting, and then it declines over a period of time. Studies have shown that GIC picks up fluoride from the oral cavity, but as the restoration ages, the ability diminishes over time [See: Sidhu S. et al., A Review of Glass-Ionomer Cements for Clinical Dentistry, J Funct Biomater, 2016, 28, 7, 3, 16]. Despite all these advantages of GIC, there are still difficulties in determining its enhanced perfomance in protection against caries. Hafshejani and coworkers [See: Hafshejani T. et al., Antibacterial glass-ionomer cement restorative materials: A critical review on the current status of extended release formulations, Journal of Controlled Release: Official Journal of the Controlled Release Society, 2017, 262, 317-328] mentioned that fluoride release in GIC possibly stops after complete setting, and Loyola-Rodriguez and coworkers [See: Loyola-Rodriguez J. et al., Growth inhibition of glass ionomer cements on mutans streptococci, Pediatr Dent, 1994, 16, 5, 346-349] mentioned that complete bacterial eradication was not detected when GIC was used. Various studies used different antibacterial agents incorporated with GIC to enhance the antibacterial effect. For example, chlorhexidine (CHX) is one of the most popular broad-spectrum antibacterial agents known for its low toxicity and the ability to protect the oral cavity against many types of bacterial presence [See: Baehni P. et al., Anti-plaque agents in the prevention of biofilm-associated oral diseases, Oral Dis, 2003, 9, Suppl 1, 23-29]. CHX has been tested in combination with GIC and showed fluoride release and antibacterial effect when CHX gluconate and CHX HMP (hexametaphosphate) were mixed with conventional GIC with concentrations of 0.25%, 0.75%, and 1.5%. Furthermore, higher concentration of CHX of both types had increased the fluoride release [See: Bellis C., et al., Glass ionomer cements functionalised with a concentrated paste of chlorhexidine hexametaphosphate provides dose-dependent chlorhexidine release over at least 14 months, J Dent, 2016, 45, 53-58].

Currently, silver particles are used to improve the antibacterial effect. The use of nano-silver in combination with antibacterial monomers enhances the antibacterial activity. Nano-silver particles are effective, including in long-distance bacterial growth. The antibacterial effect of silver in nano formation has shown its efficacy in the prevention of secondary caries and multiple species of microorganisms such as S. mutans, E. fecalis, and S. aureus [See: Kadkhoda Z. et al., Antimicrobial effect of chlorhexidine on Aggregatibacter actinomycetemcomitans biofilms associated with peri-implantitis, J Dent Res Dent Clin Dent Prospects, 2016, 10, 3, 176-180].

However, adding various materials inside GIC has weakened the mechanical properties. One of the materials used is sodium hexametaphosphate (SHMP), which has an antibacterial effect and can disintegrate dentinal inorganic material that can occlude the dentinal tubules, and therefore can work as a desensitizing agent [See: Abdallah S. et al., Release characterization and biological effect of Glass Ionomer Functionalized with Two Different Chlorohexidine Derivatives, Egyptian Dental Journal, 2021, 67, 2, 1371-1383]. Some research works have also discussed the bleaching effect of SHMP when combined with a restorative material such as resins [See: Bellis C., et al., Glass ionomer cements functionalised with a concentrated paste of chlorhexidine hexametaphosphate provides dose-dependent chlorhexidine release over at least 14 months, J Dent, 2016, 45, 53-58 and Hook, E., et al., Development of a novel antimicrobial-releasing glass ionomer cement functionalized with chlorhexidine hexametaphosphate nanoparticles, J Nanobiotechnology, 2014, 12, 3]. SHMP also has a potential to enhance the chemo-mechanical properties in restorative materials. However, SHMP liquifies the restoration matrix, which hinders the mechanical properties and setting time in GIC.

Accordingly, one object of the present disclosure is to provide a cariostatic restoration composition and a method of SHMP delivery system inside silica mesoporous nanoparticles for better SHMP release, preservation of antibacterial effect, and mechanical properties.

### SUMMARY

In an exemplary embodiment, a cariostatic restoration composition is described. The composition includes TiZSM-5 nanoparticles, where the TiZSM-5 nanoparticles are mesoporous and includes surfaces and mesopores. The composition further includes silver, sodium hexametaphosphate, and a glass ionomer cement. The silver is disposed on the surfaces and impregnated into the mesopores, and the sodium hexametaphosphate is disposed on the surfaces and impregnated into the mesopores. The amount of sodium hexametaphosphate is in a range from 40 wt. % to 160 wt. % of a weight of the TiZSM-5 nanoparticles and the amount of silver is in a range from 2 wt. % to 6 wt. % of the total weight of TiZSM-5 nanoparticles. The combined amount of sodium hexametaphosphate, silver, and TiZSM-5 nanoparticles is in a range from 0.1 wt. % to 10 wt. % of a weight of the glass ionomer cement.

In some embodiments, the amount of sodium hexametaphosphate is in a range from 60 wt. % to 140 wt. % of the weight of the TiZSM-5 nanoparticles.

In some embodiments, the amount of sodium hexametaphosphate is in a range from 75 wt. % to 125 wt. % of the weight of the TiZSM-5 nanoparticles.

In some embodiments, the amount of sodium hexametaphosphate is in a range from 90 wt. % to 110 wt. % of the weight of the TiZSM-5 nanoparticles.

In some embodiments, the combined amount of sodium hexametaphosphate and TiZSM-5 nanoparticles is in a range from 0.5 wt. % to 7 wt. % of the weight of the glass ionomer cement.

In some embodiments, the combined amount of sodium hexametaphosphate and TiZSM-nanoparticles is in a range from 1 wt. % to 5 wt. % of the weight of the glass ionomer cement.

In some embodiments, the amount of silver is in a range from 3 wt. % to 5 wt. % of the total weight of TiZSM-5 nanoparticles.

In some embodiments, the amount of silver is in a range from 3.5 wt. % to 4.5 wt. % of the total weight of TiZSM-5 nanoparticles.

In some embodiments, the amount of silver is 4 wt. % of the total weight of TiZSM-5 nanoparticles.

In some embodiments, the amount of sodium hexametaphosphate is in a range from 90 wt. % to 110 wt. % of the weight of TiZSM-5 nanoparticles, and the combined amount of sodium hexametaphosphate and TiZSM-5 nanoparticles is in a range from 1 wt. % to 5 wt. % of the weight of the glass ionomer cement.

In another exemplary embodiment, a method for cariostatic restoration is described. The method includes applying the cariostatic restoration composition to a dental caries, and curing the composition, where sodium hexametaphosphate is delivered to the dental caries.

In some embodiments, the composition includes sodium hexametaphosphate in a range from 60 wt. % to 140 wt. % of the weight of the TiZSM-5 nanoparticles.

In some embodiments, the composition includes sodium hexametaphosphate in a range from 75 wt. % to 125 wt. % of the weight of the TiZSM-5 nanoparticles.

In some embodiments, the composition includes sodium hexametaphosphate in a range from 90 wt. % to 110 wt. % of the weight of the TiZSM-5 nanoparticles.

In some embodiments, the composition includes a combined amount of sodium hexametaphosphate and TiZSM-5 nanoparticles in a range from 0.5 wt. % to 7 wt. % of the weight of the glass ionomer cement.

In some embodiments, the composition includes a combined amount of sodium hexametaphosphate and TiZSM-5 nanoparticles in a range from 1 wt. % to 5 wt. % of the weight of the glass ionomer cement.

In some embodiments, the composition includes silver in a range from 3 wt. % to 5 wt. % of the total weight of TiZSM-5 nanoparticles.

In some embodiments, the composition includes silver in a range from 3.5 wt. % to 4.5 wt. % of the total weight of TiZSM-5 nanoparticles.

In some embodiments, the composition includes silver at 4 wt. % of the total weight of TiZSM-5 nanoparticles.

In some embodiments, the composition includes sodium hexametaphosphate in a range from 90 wt. % to 110 wt. % of the weight of TiZSM-5 nanoparticles, and the composition includes a combined amount of sodium hexametaphosphate and TiZSM-5 nanoparticles in a range from 1 wt. % to 5 wt. % of the weight of the glass ionomer cement.

The foregoing general description of the illustrative embodiments and the following detailed description thereof are merely exemplary aspects of the teachings of this disclosure, and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 is a schematic flow chart of a method for cariostatic restoration, according to certain embodiments.

FIG. 2 is an x-ray diffraction (XRD) pattern showing decrease in frequencies compared to control groups, according to certain embodiments.

FIG. 3 is scanning electron microscope (SEM) images showing presence of sodium hexametaphosphate (SHMP) on zeolite nanoparticles, according to certain embodiments.

FIG. 4 is an elemental mapping showing presence of sodium (Na), and phosphate (P) m and silicon dioxide (SiO₂) inside the nanoparticle, according to certain embodiments.

FIG. 5A is a graphical representation showing surface roughness of samples before and after polishing thereof, according to certain embodiments.

FIG. 5B is a three dimensional (3D) plot showing surface roughness of the samples, according to certain embodiments.

### DETAILED DESCRIPTION

In the drawings, like reference numerals designate identical or corresponding parts throughout the several views. Further, as used herein, the words “a”, “an” and the like generally carry a meaning of “one or more”, unless stated otherwise.

Furthermore, the terms “approximately,” “approximate”, “about” and similar terms generally refer to ranges that include the identified value within a margin of 20%, 10%, or preferably 5%, and any values therebetween.

As used herein, the term “curing” refers to the process of hardening dental materials, such as fillings, sealants, or bonding agents, using light or chemicals to achieve a solid and durable state. Hardening may include the formation of covalent (polymerization and/or crosslinking) and/or ionic bonds, and may include formation of inorganic ordered structures.

A weight percent of a component, unless specifically stated to the contrary, is based on the total weight of the formulation or composition in which the component is included. For example, if a particular element or component in a composition or article is said to have 5 wt. %, it is understood that this percentage is in relation to a total compositional percentage of 100 wt. %.

As used herein, the term “zeolitic material” refers to a material having the crystalline structure or three-dimensional framework of, but not necessarily the elemental composition of, a zeolite. Zeolites are porous silicate or aluminosilicate minerals that occur in nature. Elementary building units of zeolites are SiO₄ (and if appropriate, AlO₄) tetrahedra. Adjacent tetrahedra are linked at their corners via a common oxygen atom, which results in an inorganic macromolecule with a three-dimensional framework (frequently referred to as the zeolite framework). The three-dimensional framework of a zeolite also includes channels, channel intersections, and/or cages having dimensions in the range of 0.1-10 nanometers (nm), preferably 0.2-5 nm, more preferably 0.2-2 nm. Water molecules may be present inside these channels, channel intersections, and/or cages. Zeolites that are devoid of aluminum may be referred to as “all-silica zeolites” or “aluminum-free zeolites.” Some zeolites that are substantially free of, but not devoid of, aluminum is called “high-silica zeolites.” Sometimes, the term “zeolite” is used to refer exclusively to aluminosilicate materials, excluding aluminum-free zeolites or all-silica zeolites.

In some embodiments, the zeolitic material has a three-dimensional framework that is at least one zeolite framework selected from the group consisting of a 4-membered ring zeolite framework, a 6-membered ring zeolite framework, a 10-membered ring zeolite framework, and a 12-membered ring zeolite framework. The zeolite may have a natrolite framework (e.g. gonnardite, natrolite, mesolite, paranatrolite, scolecite, and tetranatrolite), edingtonite framework (e.g. edingtonite and kalborsite), thomsonite framework, analcime framework (e.g. analcime, leucite, pollucite, and wairakite), phillipsite framework (e.g. harmotome), gismondine framework (e.g. amicite, gismondine, garronite, and gobbinsite), chabazite framework (e.g. chabazite-series, herschelite, willhendersonite, and SSZ-13), faujasite framework (e.g. faujasite-series, Linde type X, and Linde type Y), mordenite framework (e.g. maricopaite and mordenite), heulandite framework (e.g. clinoptilolite and heulandite-series), stilbite framework (e.g. barrerite, stellerite, and stilbite-series), brewsterite framework, or cowlesite framework. In some embodiments, the porous silicate and/or aluminosilicate matrix is a zeolitic material having a zeolite framework selected from the group consisting of ZSM-5, ZSM-8, ZSM-11, ZSM-12, ZSM-18, ZSM-23, ZSM-35, and ZSM-39.

Aspects of the present disclosure are directed to a system for enhancing the properties of glass ionomer cements (GIC) by incorporating sodium hexametaphosphate (SHMP) into silica nanoparticles, specifically using titanium-incorporated ZSM-5 zeolite (4 wt % Ag/TiZSM-5). The present disclosure addresses the limitations of prior art, which included issues with the burst release of fluoride and the detrimental effects on mechanical properties when combining chlorhexidine (CHX) with SHMP nanoparticles. By utilizing the present nanocomposite, the new system not only improves fluoride release but also enhances antibacterial activity, particularly against Streptococcus mutans, without compromising the mechanical integrity of the GIC. The solution demonstrates a more controlled and effective drug release profile, overcoming the drawbacks of previous formulations.

A cariostatic restoration composition (also referred to as composition) is described. The cariostatic restoration composition refers to the dental material or restorative system designed to actively prevent the formation or progression of dental caries (tooth decay) in the treated area. These materials typically contain components that release therapeutic agents, such as fluoride, which inhibit the growth of cariogenic bacteria and promote remineralization of the enamel.

The composition includes TiZSM-5 nanoparticles (titanium-incorporated ZSM-5 zeolite nanoparticles). The TiZSM-5 nanoparticles are mesoporous and include surfaces and mesopores. The mesoporous nature of TiZSM-5 nanoparticles, combined with the incorporation of titanium, enhances the material's surface area, adsorption capacity, and reactivity, making it highly suitable for applications requiring controlled release, catalysis, and improved antibacterial properties. In one or more embodiments, the average size of the TiZSM-5 nanoparticles is in a range from 300 to 1,500 nm, from 400 to 1.00 nm, preferably from 500 to 1,000 nm.

The composition further includes silver, preferably incorporated as a silver salt more preferably in the form of silver metal nanoparticles. Suitable examples of the silver salts used in dental compositions for treating dental caries may include silver nitrate, silver fluoride, silver diamine fluoride, silver sulfadiazine, silver chloride, silver bromide, silver iodide, silver sulfate, silver acetate, silver oxide, silver carbonate, silver phosphate, silver citrate, silver lactate, silver tartrate, silver formate, silver picolinate, silver benzoate, silver gluconate, silver salicylate, silver nitrate solution, silver fluoride solution, silver peroxide, silver proteinate, silver tetraborate, silver aluminate, silver metaphosphate, silver polyphosphate, silver chloride complexes, and silver glycinate. In a preferred embodiment, silver salt is silver nitrate.

The silver is disposed on the surfaces and impregnated into the mesopores. The silver is applied as a thin layer or nanoparticles on the surface of the material. The surfaces are coated/deposited wholly/partially in a uniform and continuous manner. In an embodiment, the silver forms a monolayer on the surfaces. In another embodiment, the silver may include more than a single layer on the surfaces. The silver on the surfaces can release silver ions (Ag⁺), which are toxic to bacteria. This slow release helps in continuously fighting bacteria, particularly including Streptococcus mutans. The amount of silver is in a range from 0.5 to 10 wt. %, preferably 2 to 6 wt. %, preferably 3 to 5 wt. %, preferably 3.5 to 4.5 wt. % of the total weight of TiZSM-5 nanoparticles. In a preferred embodiment, the amount of silver in the composition is about 4 wt. % of the total weight of TiZSM-5 nanoparticles.

The composition further includes sodium hexametaphosphate. The sodium hexametaphosphate is disposed on the surfaces and impregnated into the mesopores, and may be homogeneously distributed through the zeolite and bonded only to the surface of the Ag. The disposal of sodium hexametaphosphate (SHMP) on surfaces and its impregnation into the mesopores of a material allows for a synergistic approach to caries prevention and remineralization. SHMP acts as both a surface treatment to provide immediate protection and a reservoir for sustained release of phosphate ions, leading to continuous remineralization, inhibition of demineralization, and enhanced resistance to bacterial attack. This dual action is particularly beneficial in dental restorations, sealants, and cements, improving their performance and longevity in maintaining oral health. The amount of sodium hexametaphosphate is in a range from 20 to 180 wt. %, 40 to 160 wt. %, preferably 60 to 140 wt. %, preferably 75 to 125 wt. %, preferably 90 to 110 wt. %, of the weight of the TiZSM-5 nanoparticles.

The composition includes a glass ionomer cement. Glass ionomer cements (GICs) are dental materials composed of a silicate glass powder and a polyacid-based liquid that undergoes an acid-base reaction to form a hardened material. These cements are characterized by their ability to bond chemically to both dental hard tissues and metals. The setting reaction involves the release of fluoride ions, which can contribute to the prevention of tooth decay, making GICs cariostatic. Additionally, GICs can release fluoride over time, although the release rate typically diminishes after the initial burst during setting. GICs are used for various dental applications, including restorative fillings, luting cements, and lining materials, due to their favorable biocompatibility, ease of manipulation, and adhesive properties.

In some embodiments, the combined amount of sodium hexametaphosphate and TiZSM-5 nanoparticles is in a range from 0.1 to 15 wt. %, 0.5 to 7 wt. %, preferably 1 to 5 wt. %, of the weight of the glass ionomer cement. In some embodiments, the amount of sodium hexametaphosphate is in a range from 90 to 110 wt. % of the weight of TiZSM-5 nanoparticles, and the combined amount of sodium hexametaphosphate and TiZSM-5 nanoparticles is in a range from 1 to 5 wt. % of the weight of the glass ionomer cement. In some embodiments, the combined amount of sodium hexametaphosphate, silver, and TiZSM-5 nanoparticles is in a range from 0.1 to 10 wt. % of the weight of the glass ionomer cement.

In some embodiments, the composition may include, but is not limited to, other components such as resins (such as Bis-GMA or TEGDMA), glass fillers (like silica or quartz particles), fluoride agents (such as sodium fluoride or calcium fluoride), calcium compounds (e.g., calcium hydroxide or calcium phosphate), and bonding agents (such as adhesive resins or primers). These components work together to restore the tooth structure, promote remineralization, and provide a durable bond between the filling material and the tooth surface.

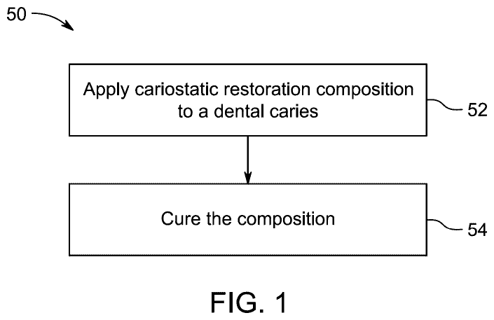

FIG. 1 illustrates a schematic flow chart of a method 50 for cariostatic restoration. The order in which the method 50 is described is not intended to be construed as a limitation, and any number of the described process steps can be combined to implement the method 50. Additionally, individual steps may be removed or skipped from the method 50 without departing from the spirit and scope of the present disclosure.

At step 52, the method 50 includes applying the composition as described above to a dental caries. Dental caries, or tooth decay, is a progressive breakdown of tooth structure caused by the interaction between bacteria in the mouth and fermentable carbohydrates like sugars. Bacteria metabolize sugars to produce acids that demineralize tooth enamel, leading to the formation of cavities. If untreated, the decay can spread to the underlying dentin and pulp, causing pain, sensitivity, and infection. Factors in the development of caries may include bacterial biofilm, poor oral hygiene, diet high in sugars, and reduced saliva flow.

In some embodiments, dental caries may be caused by bacteria such as Streptococcus mutans, Streptococcus sobrinus, Lactobacillus acidophilus, Lactobacillus casei, Lactobacillus rhamnosus, Lactobacillus fermentum, Lactobacillus plantarum, Lactobacillus reuteri, Lactobacillus johnsonii, Lactobacillus salivarius, Actinomyces naeslundii, Actinomyces viscosus, Actinomyces israelii, Actinomyces odontolyticus, Actinomyces meyeri, Actinomyces naeslundii, Bifidobacterium bifidum, Bifidobacterium adolescentis, Bifidobacterium longum, Bifidobacterium dentium, Veillonella parvula, Veillonella dispar, Veillonella atypica, Corynebacterium matruchotii, Corynebacterium diphtheriae, Corynebacterium pseudodiphtheriticum, Corynebacterium amycolatum, Fusobacterium nucleatum, Fusobacterium necrophorum, Fusobacterium periodonticum, Prevotella intermedia, Prevotella melaninogenica, Prevotella denticola, Prevotella oris, Eubacterium nodatum, Eubacterium brachy, Eubacterium timidum, Eubacterium saphenum, Granulicatella adiacens, Granulicatella elegans, Bacteroides fragilis, Bacteroides thetaiotaomicron, Bacteroides ovatus, Bacteroides vulgatus, Bacteroides caccae, Streptococcus salivarius, Streptococcus anginosus, Streptococcus sanguinis, Streptococcus oralis, Streptococcus gordonii, Streptococcus mitis, Streptococcus pneumoniae, Streptococcus pyogenes, Streptococcus agalactiae, Enterococcus faecalis, Enterococcus faecium, Enterococcus durans, Enterococcus casseliflavus, Streptococcus equinus, Streptococcus uberis, Streptococcus porcinus, Streptococcus canis, Streptococcus suis, Streptococcus bovis, Streptococcus lutetiensis, Streptococcus parauberis, Streptococcus constellatus, Streptococcus mutans biofilm-forming strains, Streptococcus sobrinus biofilm-forming strains, Rothia mucilaginosa, Rothia dentocariosa, Rothia aeria, Rothia nasimurium, Moraxella catarrhalis, Moraxella lacunata, Moraxella osloensis, Neisseria subflava, Neisseria sicca, Neisseria elongata, Neisseria mucosa, Neisseria gonorrhoeae, Propionibacterium acnes, Propionibacterium propionicum, Propionibacterium jensenii, Propionibacterium freudenreichii, Propionibacterium avidum, Clostridium perfringens, Clostridium tetani, Clostridium difficile, Clostridium sordellii, Clostridium baratii, Clostridium novyi, Clostridium botulinum, Clostridium beijerinckii, Pseudomonas aeruginosa, Pseudomonas putida, Pseudomonas stutzeri, Pseudomonas citronellolis, Pseudomonas fluorescens, Pseudomonas mendocina, Salmonella enterica, Salmonella typhi, Salmonella choleraesuis, Salmonella enteritidis, Salmonella paratyphi, Escherichia coli, Escherichia coli 0157: H7, Escherichia coli K12, Escherichia fergusonii, Escherichia albertii, Klebsiella pneumoniae, Klebsiella oxytoca, Klebsiella planticola, Klebsiella aerogenes, Acinetobacter baumannii, Acinetobacter calcoaceticus, Acinetobacter lwoffii, Acinetobacter junii, Shigella dysenteriae, Shigella flexneri, Shigella boydii, Shigella sonnei, Bifidobacterium breve, Bifidobacterium pseudocatenulatum, Bifidobacterium lactis, Bifidobacterium kashiwanohense, Streptococcus thermophilus, Streptococcus salivarius, Streptococcus constellatus, Fusobacterium nucleatum subsp. vincentii, Fusobacterium nucleatum subsp. fusiforme, Fusobacterium mortiferum, Fusobacterium necrophorum subsp. necrophorum, Fusobacterium necrophorum subsp. funduliforme, Prevotella bivia, Prevotella buccalis, Prevotella denticola, Prevotella disiens, Prevotella melaninogenica, Prevotella oralis, Prevotella nigrescens, Prevotella intermedia, Porphyromonas gingivalis, Porphyromonas endodontalis, Porphyromonas asaccharolytica, Porphyromonas macacae, Bacteroides fragilis, Bacteroides vulgatus, Bacteroides thetaiotaomicron, Bacteroides ovatus, Bacteroides caccae, Campylobacter rectus, Campylobacter showae, Campylobacter concisus, Campylobacter curvus, Leptotrichia buccalis, Leptotrichia maltophilia, Leptotrichia hofstadii, Leptotrichia wadei, Tannerella forsythia, Tannerella pectinovora, Tannerella denticola, Tannerella saccharolytica, Simonsiella spp., Selenomonas spp., Fusobacterium nucleatum subsp. necrophorum, Atopobium vaginae, Atopobium rimae, Anaeroglobus geminatus, Dialister invisus, Dialister pneumosintes, Megasphaera elsdenii, Megasphaera micronuciformis, Megasphaera aemilioides, Eubacterium saburreum, Eubacterium yurii, Lactobacillus gasseri, Lactobacillus helveticus, Lactobacillus delbrueckii. In a preferred embodiment, the dental caries is caused by Streptococcus mutans. Tools such as microbrushes, cotton pellets, syringes, applicator tips, spatulas, dental brushes, application trays, filling instruments (plastic fillers, condensers), carving instruments, ultrasonic scalers, etching tools, cotton rolls and gauze, endodontic applicator, laser handpieces may be used to apply the composition to the dental caries.

At step 54, the method 50 includes curing the composition. As used herein, the term ‘curing’ refers to the process of hardening dental materials, such as fillings, sealants, or bonding agents, using light or chemicals to achieve a solid and durable state. After curing, the composition treats the applied area and helps prevents further carries from forming through the release of cariostatic compounds.

### EXAMPLES

The following examples demonstrate a cariostatic restoration composition. The examples are provided solely for illustration and are not to be construed as limitations of the present disclosure, as many variations thereof are possible without departing from the spirit and scope of the present disclosure.

### Example 1: Sample Calculation

The sample size for the present disclosure was calculated using the results from previous research work. G*power 3.1.9.4 software was used for calculation. The predicted sample for each group was a total of 5 (n=5) samples, while keeping the level of significance at 0.05, the effect size at 0.92, and the power at 95%.

### Example 2: Statistical Analysis

The obtained data was checked for normality using Shapiro Wilk's test and a p-value greater than 0.05 was considered as a normal data distribution. Descriptive statistics were presented using frequencies, percentages, mean, and standard deviation as appropriate. For comparison between the samples, a one-way ANOVA/Kruskal Wallis Test followed by post hoc Tukey's was performed, and a p-value of less than 0.05 was considered. The data analysis was performed using SPSS (statistical package for social science) version 24, IBM, USA for desktop.

### Example 3: Silver Impregnated on TiZSM-5 (4 wt. % Ag/TiZSM-5)

Fifty grams of TiZSM-5 zeolite nanoparticle was acquired from (Euro support manufacturing Czechia, s.r.o, Zaluz, 1,43670 Litvino, Ti-ZSM-5). 0.0126 g of silver nitrate was taken and dissolved in 60 mL of water, to which was added 2 g of TiZSM-5 and stirred overnight, and dried at 100° C. for 6 h. The sample was then calcined at 550° C. for 3 h.

### Example 4: Mixing Sodium Hexametaphosphate (SHMP) and 4 wt. % Ag/TiZSM-5

SHMP delivery method was performed using the incipient wetness impregnation technique. 1 g of SHMP and 1 g of 4 wt. % Ag/TiZSM-5 were hydrated with drops of distilled water until the mixture became a paste and mixed at a 1:1 ratio by weight in a mortar and pestle for 10 min. After the mixture became homogenous, the mixture was kept in an oven at 60° C. for a night to dry. Further, samples were collected and ground to powder form for use.

### Example 5: Mixing SHMP+4 wt. % Ag/TiZSM-5 with Glass Ionomer Cements (GIC) (1%, 2% and 5%)

A composition of 0.05 g of SHMP and 4 wt. % Ag/TiZSM-5 nanocomposite was added to 5 g of GIC powder (1%) and mixed by dry mixing method using mortar and pestle. The ratio was varied to 2% and 5% using 0.1 g and 0.25 g of SHMP+4 wt. % Ag/TiZSM-5 with 5 g of GIC powder.

### Example 6: SEM/EDX (Elemental Mapping)

After the collection of functionalized mesoporous silica nanoparticles with SHMP, samples were prepared for scanning electron microscope (SEM) to detect the surface coverage of SHMP salts on the zeolite MSN@, and energy dispersive x-ray (EDX) elemental mapping was conducted to detect the presence of SiO₂, phosphate, sodium silver, and titanium inside the nanoparticles.

### Example 7: Antimicrobial efficacy

GIC discs were prepared (n=7) for each sample. A 50 μL suspension of Streptococcus mutans suspension, cultured in BHI (Becton Dickinson) broth adjusted to 103 colony-forming units (CFU)/mL, was placed on top of each cured GIC disc then stored in an anaerobic incubator at 37° C. for 18 hours. Then, each cultured disc was immersed in 9.95 mL of 0.01 M phosphate-buffered saline (PBS; pH 7.4) and vigorously shaken, then diluted with PBS and inoculated on BHI agar plates. Plates were then stored in an anaerobic incubator at 37° C. for 48 hours, after which the bacterial count was calculated.

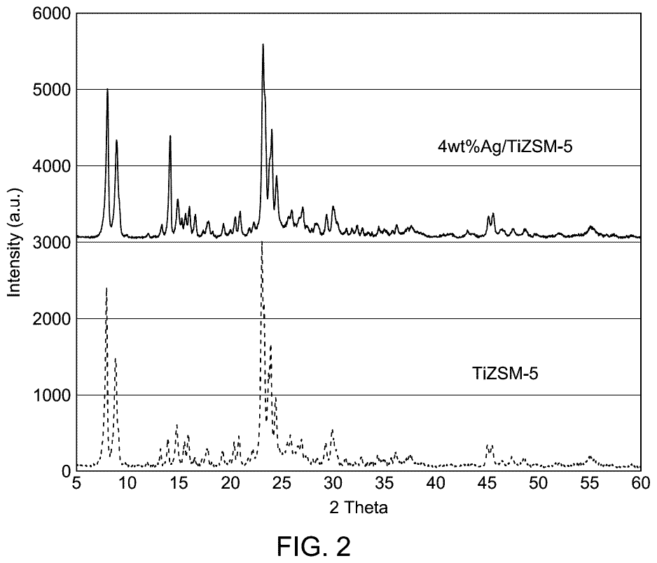

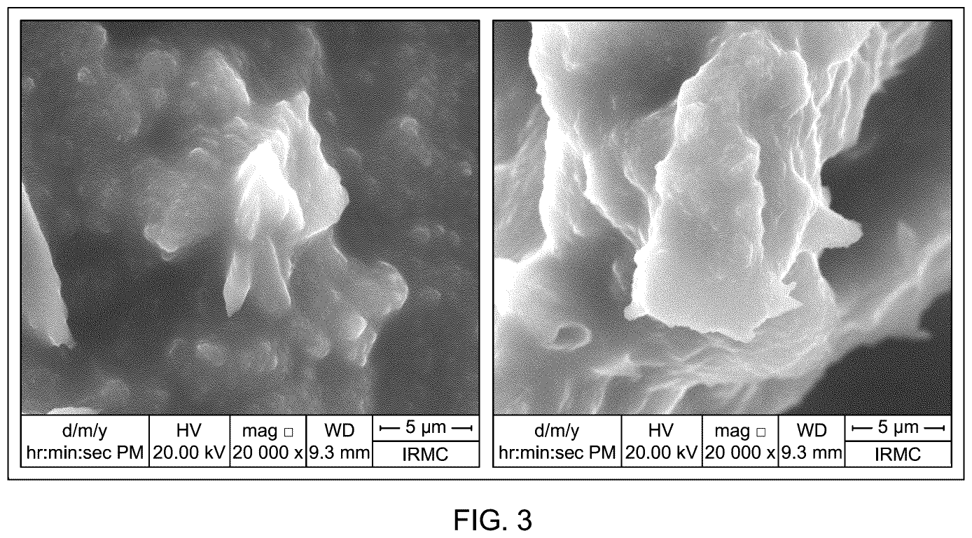

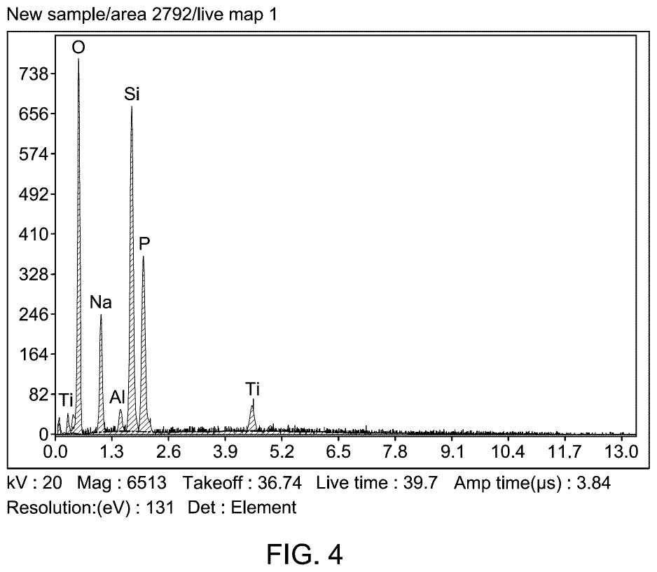

Two samples, such as Z-MSN@Ag and Z-MSN@Ag/Ti functionalized with the SHMP, were tested in X-ray diffraction (XRD) spectroscopy to detect frequency of materials present and were compared with plain Z-MSN@ to measure the difference in frequencies, as shown in FIG. 2. SEM has shown that SHMP salt was present on top of nanoparticles. As illustrated in FIG. 3, presence of SHMP was recorded upon imaging. As shown in FIG. 4, elemental mapping has proved the presence of sodium and phosphate inside the nanoparticle. Moreover, silica oxide and titanium were not affected by the presence of SHMP.

Antibacterial test was conducted against one strain of bacterial colony S. Mutans, in an agar diffusion plate using sheep brain. The One-way ANOVA did not show statistical difference between the groups that were tested. GIC, titanium zeolite, and sliver titanium zeolite non functionalized were highest among the groups with antibacterial zones of inhibition. SHMP, when tested, showed that as the percentage of concentrations increases the antibacterial efficacy also increases, as seen in Table 1.

TABLE 1
Statistical data of various sample groups.

| Groups | Mean | Std. Deviation | p-value |
|---|---|---|---|
| Control | 3.33 | 5.77 | 0.055 |
| TiZSM-5 | 10.00 | 0.00 | |
| 4 wt % Ag/TiZSM-5 | 0.00 | 0.00 | |
| Ag | 130.00 | 225.17 | |
| ZHX 1% | 810.00 | 523.07 | |
| ZHX 2% | 1270.00 | 1206.52 | |
| ZHX 5% | 246.67 | 297.71 | |

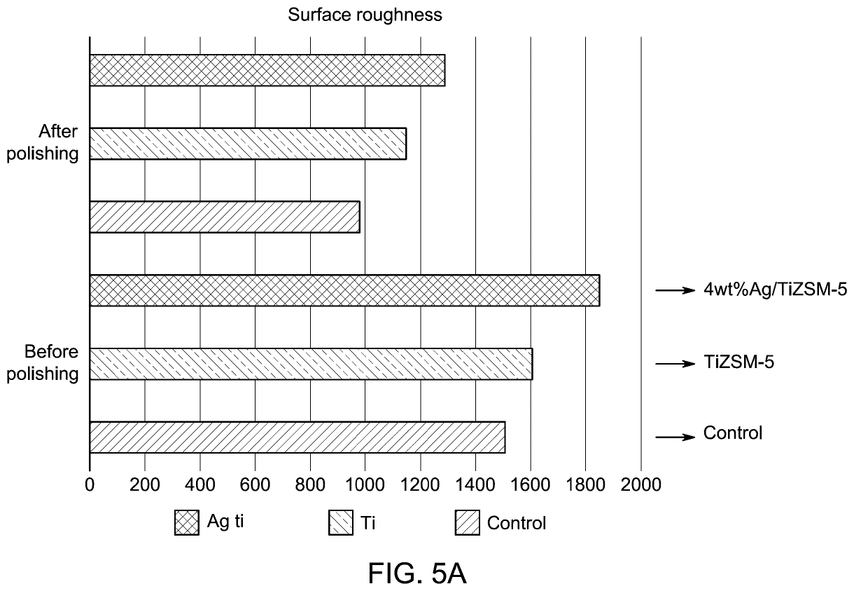

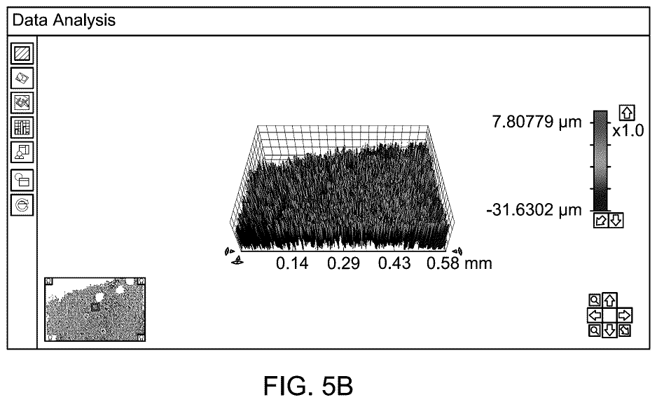

Referring to FIGS. 5A-5B, three samples were tested for surface roughness, and it has shown a slight increase in surface roughness mainly before polishing. After polishing, a noticeable decrease in roughness was recorded.

According to the present disclosure, an impregnation technique for SHMP to 4 wt. % Ag impregnated on TiZSM-5 zeolite nanoparticles was developed. The phase of silver nanoparticles and microporous zeolite (TiZSM-5) was successfully recorded using X-ray diffraction analysis, electron transmission microscopy, scanning electron microscopy, and elemental mapping. The functionalization process aimed to enhance the antibacterial effect of GIC and increase the mechanical properties of said material.

The surface analysis of 4 wt. % Ag impregnated on TiZSM-5 revealed that phosphate and sodium were present in X-ray diffraction analysis, nonetheless, on the SEM images, salts were present on the surface of nanoparticles. Ge and coworkers [See: Ge K., et al., A novel glass ionomer cement with silver zeolite for restorative dentistry, J Dent, 2023, 133, 104524] has found that silver-loaded zeolite has similar surface morphology as GIC [See: Krzyściak W., et al., The virulence of Streptococcus mutans and the ability to form biofilms, Eur J Clin Microbiol Infect Dis, 2014, 33, 4, 499-515]. Furthermore, surface roughness indicates that the presence of SHMP inside the nanoparticle did not affect the surface roughness or the mechanical properties such as setting time.

The most common bacteria in the oral cavity is Streptococcus mutans. A single type of bacteria was tested using an agar plate and brain broth. The disadvantage of this methodology is that the complexity of bacteria and fungi varies in the oral cavity. The antibacterial effect of GIC against Streptococcus mutans was high compared to SHMP alone; the growth inhibition was similar to Rodriguez and coworkers [See: Loyola-Rodriguez J. et al., Growth inhibition of glass ionomer cements on mutans streptococci, Pediatr Dent, 1994, 16, 5, 346-349]. GIC had shown a successful S. mutans growth inhibition compared to the different kinds of bacteria. The antibacterial effect of SHMP, when tested alone in an agar plate, was found ineffective in growth inhibition in low concentrations; however, when 3-5% of Ag was used, greater inhibition areas were recorded. Hosida and coworkers [See: Hosida T., et al., Effects of Sodium Hexametaphosphate and Fluoride on the pH and Inorganic Components of Streptococcus mutans and Candida albicans Biofilm after Sucrose Exposure, Antibiotics (Basel), 2022, 11, 8, 1044] had found that when SHMP was used in low concentrations, it had an effect on bacterial colonies; however, as the concentration increases, the antibacterial effect along with fluoride increases. Silver and silver/titanium groups loaded nanoparticles, when tested against S. mutans, were found to have a potent reactivity with bacterial growth. The present disclosure has shown the highest antibacterial activity within Ag and 4 wt. % Ag impregnated on TiZSM-5 test groups compared to the control. Ge and coworkers tested the bacterial growth and kinetics and found that groups with silver zeolite had less bacterial growth over a 7-day period than the control group. Surface-attached bacteria had more dead cells upon immunofluorescence imaging compared to control. In the present disclosure, SHMP was impregnated, and no study was carried out on the effect of 4 wt. % Ag impregnated on TiZSM-5 zeolite-loaded SHMP NP on the surface roughness. When mixed, samples showed a rougher surface compared to the control group before polishing; however, no difference was found after polishing. No difference was found between control and zeolite silver NP. In the present disclosure, SHMP loaded 4 wt. % Ag impregnated on TiZSM-5 zeolite has shown the desired results in restorative materials. Antibacterial activity was also increased when silver and titanium were used along with the SHMP.

Numerous modifications and variations of the present disclosure are possible in light of the above teachings. It is therefore to be understood that within the scope of the appended claims, the invention may be practiced otherwise than as specifically described herein.

1. A cariostatic restoration composition, the composition comprising:
TiZSM-5 nanoparticles, wherein the TiZSM-5 nanoparticles are mesoporous and comprise surfaces and mesopores;
silver, wherein the silver is disposed on the surfaces and impregnated into the mesopores,
sodium hexametaphosphate, wherein the sodium hexametaphosphate is disposed on the surfaces and impregnated into the mesopores; and
a glass ionomer cement,
wherein the amount of sodium hexametaphosphate is in a range from 40 to 160 wt. % of the weight of the TiZSM-5 nanoparticles,
wherein the amount of silver is in a range from 2 to 6 wt. % of the total weight of TiZSM-5 nanoparticles,
wherein the combined amount of sodium hexametaphosphate, silver, and TiZSM-5 nanoparticles is in a range from 0.1 to 10 wt. % of the weight of the glass ionomer cement, and
wherein the TiZSM-5 nanoparticles have an average size in a range from 500 to 1,000 nm.

2. The composition of claim 1, wherein the amount of sodium hexametaphosphate is in a range from 60 to 140 wt. % of the weight of the TiZSM-5 nanoparticles.

3. The composition of claim 2, wherein the amount of sodium hexametaphosphate is in a range from 75 to 125 wt. % of the weight of the TiZSM-5 nanoparticles.

4. The composition of claim 3, wherein the amount of sodium hexametaphosphate is in a range from 90 to 110 wt. % of the weight of the TiZSM-5 nanoparticles.

5. The composition of claim 1, wherein the combined amount of sodium hexametaphosphate and TiZSM-5 nanoparticles is in a range from 0.5 to 7 wt. % of the weight of the glass ionomer cement.

6. The composition of claim 5, wherein the combined amount of sodium hexametaphosphate and TiZSM-5 nanoparticles is in a range from 1 to 5 wt. % of the weight of the glass ionomer cement.

7. The composition of claim 1, wherein the amount of silver is in a range from 3 to 5 wt. % of the total weight of TiZSM-5 nanoparticles.

8. The composition of claim 7, wherein the amount of silver is in a range from 3.5 to 4.5 wt. % of the total weight of TiZSM-5 nanoparticles.

9. The composition of claim 8, wherein the amount of silver is 4 wt. % of the total weight of TiZSM-5 nanoparticles.

10. The composition of claim 9, wherein the amount of sodium hexametaphosphate is in a range from 90 to 110 wt. % of the weight of TiZSM-5 nanoparticles, and the combined amount of sodium hexametaphosphate and TiZSM-5 nanoparticles is in a range from 1 to 5 wt. % of the weight of the glass ionomer cement.

11. A method for cariostatic restoration, the method comprising:
applying the composition of claim 1 to a dental caries; and
curing the composition,
wherein sodium hexametaphosphate is delivered to the dental caries.

12. The method of claim 11, wherein the composition comprises sodium hexametaphosphate in a range from 60 to 140 wt. % of the weight of the TiZSM-5 nanoparticles.

13. The method of claim 12, wherein the composition comprises sodium hexametaphosphate in a range from 75 to 125 wt. % of the weight of the TiZSM-5 nanoparticles.

14. The method of claim 13, wherein the composition comprises sodium hexametaphosphate in a range from 90 to 110 wt. % of the weight of the TiZSM-5 nanoparticles.

15. The method of claim 11, wherein the composition comprises a combined amount of sodium hexametaphosphate and TiZSM-5 nanoparticles in a range from 0.5 to 7 wt. % of the weight of the glass ionomer cement.

16. The method of claim 15, wherein the composition comprises a combined amount of sodium hexametaphosphate and TiZSM-5 nanoparticles in a range from 1 to 5 wt. % of the weight of the glass ionomer cement.

17. The method of claim 11, wherein the composition comprises silver in a range from 3 to 5 wt. % of the total weight of TiZSM-5 nanoparticles.

18. The method of claim 17, wherein the composition comprises silver in a range from 3.5 to 4.5 wt. % of the total weight of TiZSM-5 nanoparticles.

19. The method of claim 18, wherein the composition comprises silver at 4 wt. % of the total weight of TiZSM-5 nanoparticles.

20. The method of claim 19, wherein the composition comprises sodium hexametaphosphate in a range from 90 to 110 wt. % of the weight of TiZSM-5 nanoparticles, and the composition comprises a combined amount of sodium hexametaphosphate and TiZSM-5 nanoparticles in a range from 1 to 5 wt. % of the weight of the glass ionomer cement.
